(19) United States
(12) Patent Application Publication
(10) Pub. No.: US 2014/0031894 A1
(43) Pub. Date: Jan. 30, 2014

(54) METHOD AND DEVICE FOR POST-OPERATIVE APPLICATION OF PULSED RADIOFREQUENCY FOR PREVENTION OF PAIN AND CARTILAGE LOSS

(71) Applicants: Eugene G. Lipov, Chicago, IL (US); Daniel J. Solomon, Novato, CA (US)

(72) Inventors: Eugene G. Lipov, Chicago, IL (US); Daniel J. Solomon, Novato, CA (US)

(21) Appl. No.: 13/948,823

(22) Filed: Jul. 23, 2013

Related U.S. Application Data
(60) Provisional application No. 61/674,739, filed on Jul. 23, 2012.

(51) Int. Cl. A61N 1/36; A61M 1/00; A61N 1/05

(52) U.S. Cl. CPC A61N 1/36071; A61N 1/05; A61M 1/0023
USPC 607/46; 607/116

(57) ABSTRACT

A pain alleviation method as well as cartilage sparring or repairing method in the intraoperative as well as perioperative time of treatment device are disclosed herein. Pain maybe alleviated by applying a pulsed radio frequency (PRF) signal in the intraarticular as well as intrabursal or intracapsular area of a joint, while controlling the fluids in the joint area using suction. A device for carrying out the method includes a PRF electrode, a suction device, removing the fluids in the intraarticular and/or intracapsular and/or intrabursal area of a joint. The PRF electrode and suction device maybe collocated in a flexible tube that maybe applied through a cannula. PRF signal is applied to the electrode for approximately 10 minutes at 2 Hz with a pulse width of approximately 10 ms and approximately 55 V at approximately 42 degrees or similar setting.

This application claims the benefit of U.S. Provisional Application No. 61/674,739, filed Jul. 23, 2012, which is hereby incorporated by reference in its entirety.

### BACKGROUND OF THE INVENTION

This disclosure relates to pulsed radiofrequency (PRF) devices and methods to alleviate acute pain, and minimize cartilage loss caused by surgery, as well as the prevention of persistent pain and loss of cartilage in the perioperative period following surgery.

Conventional surgical techniques for treating or evaluating disorders of joints (e.g., diarthroidal joints not limited to but including shoulder, elbow, wrist, ankle, knee, and hip) include orthopedic surgery, commonly arthroscopic surgery. Arthroscopic techniques are less invasive than open surgical techniques and are the mainstay most non-arthroplasty (joint replacement) surgery today.

Arthroscopic surgery employs an arthroscope, which is a type of endoscope that is inserted into the joint through a small incision with or without a cannula (a tube that is placed into the joint first with the arthroscope subsequently placed into the joint via the tube), as shown in FIG. 1. Arthroscopy provides an advantage over traditional open surgery in that the joint does not have to be fully exposed. Instead, a surgeon typically makes two or more small incisions: one incision for the arthroscope, which includes light and video functionality for viewing the surgical site, and one incision for the surgical instruments that are used to perform the operation.

Despite the benefits of having a less-invasive surgery, arthroscopic techniques still leave a patient with inflammation (due to mechanical invasion) and pain. When a patient undergoes surgery, a variety of tissues and nerve endings may be damaged or traumatized. Various inflammatory factors and tissue signal substances are activated. This cascade of events can also lead to cartilage loss, believed to be due to inflammatory factor activation following surgery.

Further, in the immediately post-operative period, unrelieved acute surgical pain creates needless suffering along with delayed discharges from hospital/operative rooms, and increased costs of care. Additionally, some evidence suggests that poorly controlled acute postoperative pain may also be associated with increased chronic postoperative pain in the longer perioperative period and beyond.

Recent focus on acute post-operative pain has led to techniques for alleviating the acute pain due to surgery. One such approach involves a surgeon inserting a device called a “pain pump” via a catheter into the joint space (synovial space) and/or, in the case of the shoulder joint, the rotator cuff or subacromial areas. The surgeon then continuously administers pain medication (a local anesthetic), such as bupivacaine, via the catheter into the joint, for up to 20 hours after the surgery. This approach has led to good pain relief; however post-arthroscopy patients treated by pain pump have experienced loss or disintegration of cartilage. This has been shown to be significantly more than cartilage loss noted to happen after arthroscopy, possibly due to inflammation caused by surgery. This loss of cartilage can cause severe pain and loss of joint mobility and devastating arthritis.

Recently, post-operative pain and possibly minimization of cartilage loss has been improved with the use of external pulsed radiofrequency (EPRF) devices, however this device's efficacy is limited by the patient's compliance with this approach (the device must be placed in close vicinity of the joint for a number of weeks) and the low power of the pulsed device.

### Cartilage Overview

Chondrocytes are cells found in cartilage which, when they die, do not regenerate. When chondrocytes are gone, loss of cartilage matrix (chondrolysis) is pervasive and permanent. Common symptoms of chondrolysis are pain, stiffness of the joint and decreased range of motion, and decreased strength.

We believe an alternative method for alleviating pain in the postoperative period as well as preventing cartilage loss and disability following joint surgery can be achieved with the use of pulsed radiofrequency (PRF) immediately following surgery. In particular, PRF can be delivered via one or more catheters in the operating or recovery room, immediately following arthroscopy.

To optimize efficacy of PRF, excess or residual fluid that accumulates in the joint during arthroscopy (necessary to visualize the joint) must be removed. The fluid may have unpredictable effects on PRF, such as power dissipation and/or change in current flow previously reported. Further, due to the catheter being placed during surgery, cannulas used for arthroscopy can be used to confirm intraarticular placement of the catheter with visual guidance with the arthroscope.

A need exists for a method that alleviates the acute pain as well as prevents cartilage loss caused by surgery. As a goal, the new pain alleviation method should aim to deliver any one or more of the following benefits: help reduce inflammation, produce an analgesic effect, promote new blood vessel formation, reduce post-operative disability and/or prevent cartilage loss (all effects demonstrated with the use of PRF). The method should be able to be administered during an operation, or immediately after an operation, or any combination of the described times. Additionally, a medical device that enables the above without significant or common side-effects is needed.

### SUMMARY OF THE INVENTION

In one embodiment of the invention, a pain alleviation method comprises: (a) verifying the location of a PRF electrode intraarticular, intracapsular and/or intrabursal area of a joint, (b) applying a PRF signal via the PRF electrode to the intraarticular, intracapsular and/or intrabursal area of a joint, and (c) removing the fluids from the intraarticular, intracapsular and/or intrabursal area by direct suction in the area. The PRF signal maybe applied to the electrode of the device for a typical duration and pattern (approximately 10 minutes at 2 Hz with a pulse width of approximately 10 ms and approximately 55 V at approximately 42 degrees). Other efficacious settings maybe used.

In another embodiment of the invention, a pain alleviation device comprises a) a PRF electrode capable of being applied to an intraarticular, intracapsular and/or intrabursal area of a joint, and b) a suction device, wherein the suction device removes the fluid from intraarticular, intracapsular and/or intrabursal area of a joint.

The device of this embodiment may be co-located in a flexible tube structure, and may further comprise a cannula for introducing the device into an intraarticular, intracapsular and/or intrabursal area of a joint. The current invention utilizes pulsed radiofrequency during the immediate post-operative period (within minutes following surgery in the operating room or in the surgical recovery room). The patient is thus not responsible for compliance. We can apply higher power (alternating current (AC)) than is available by EPRF devices.

The suction device of this embodiment may comprise a tube with fenestrations along its length. The PRF signal may be applied to the electrode of the device for a typical duration and pattern (approximately 10 minutes at 2 Hz with a pulse width of approximately 10 ms and approximately 55 V at approximately 42 degrees). Other efficacious settings maybe used.

### BRIEF DESCRIPTION OF THE DRAWINGS

The described embodiments and other features, advantages and disclosures contained herein, and the manner of attaining them, will become apparent and the present disclosure will be better understood by reference to the following description of various exemplary embodiments of the present disclosure taken in conjunction with the accompanying drawings, wherein:

FIGS. 1A-1B illustrate an example of a joint to which an embodiment of the pain alleviation method may be administered;

FIG. 2A-2F illustrates an embodiment of a pain-alleviation method and an embodiment of a pain-alleviation device used to alleviate acute pain due to surgery of a shoulder joint;

FIG. 3 illustrates an embodiment of a pain-alleviation method that includes application of multiple PRF signals;

FIG. 4 illustrates an overview of how an embodiment of a pain-alleviation device may be integrated with a PRF generating source and a return electrode to apply embodiments of the pain-alleviation method described herein;

FIG. 5A-5D illustrate embodiments of a pain-alleviation device and some of its components.

Like reference numerals indicate the same or similar parts throughout the several figures.

An overview of the features, functions and configuration of the components depicted in the various figures will now be presented. It should be appreciated that not all of the features of the components of the figures are necessarily described. Some of these non-discussed features, such as various sizes, thickness, widths, materials, etc., as well as discussed features are inherent from the figures. The drawings are not drawn to scale. Other non-discussed features may be inherent in component geometry or configuration.

### DETAILED DESCRIPTION OF THE INVENTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

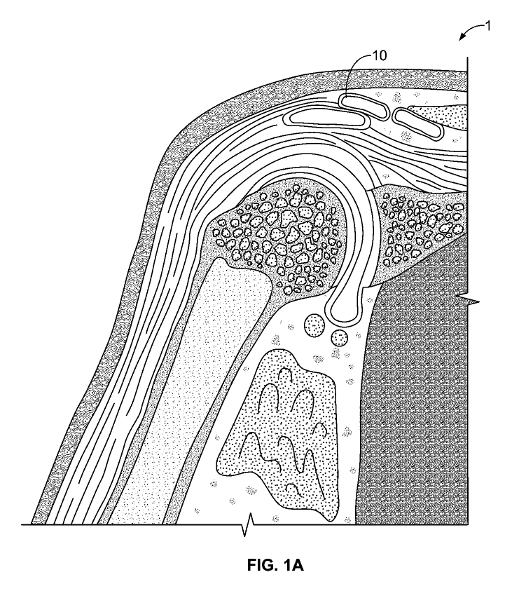

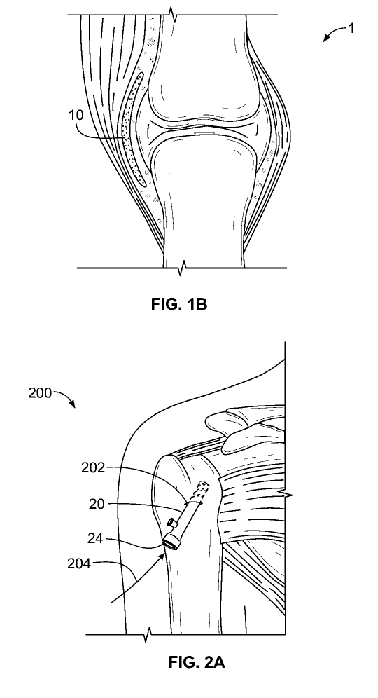

The disclosure of the present application provides a pain alleviation method 200 that may be used to reduce acute pain that is caused from surgery. Embodiments of the pain-alleviation method 200 and device 50 may be used to treat joints (e.g., diarthrodial joints, not limited to but including shoulder, elbow, wrist, ankle, knee, and hip), such as the shoulder joint as illustrated in FIG. 1A, and the knee joint as illustrated in FIG. 1B.

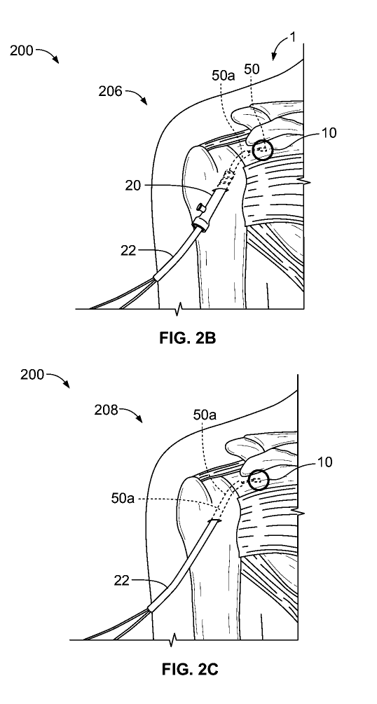

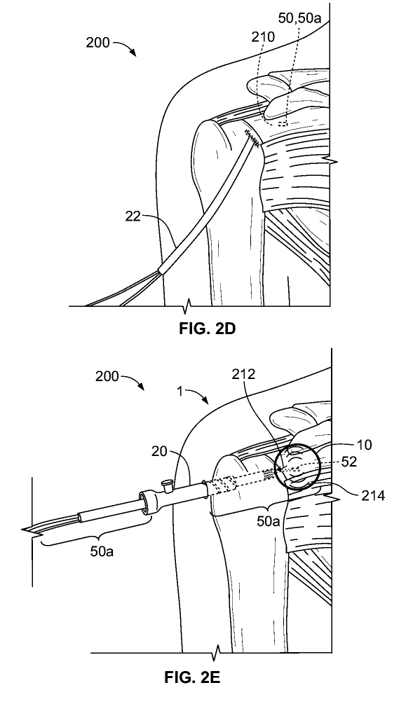

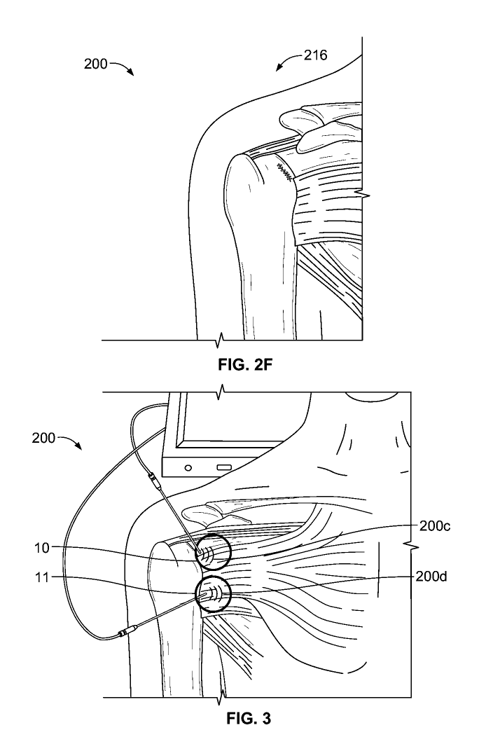

Embodiments of the pain-alleviation method 200 and device 50 may be applied at any one or more of the following times: before surgery, during surgery, after surgery. For example, the embodiment of the pain alleviation method, as illustrated in FIGS. 2A-2F, maybe applied a short time after surgery.

As illustrated in FIGS. 2A-2F, the pain alleviation method 200 may simply include applying a pulsed radio frequency (PRF) signal to an intrabursal area 10 of a joint 1 (see FIG. 2F and FIGS. 1A, 1B). PRF signal application may achieve pain relief by delivering strong electric fields and heat bursts with tip temperatures not exceeding 42 degrees Celsius. PRF has been shown to have negligible neurodestructive characteristics permitting its safe application to both peripheral nerves because it does not cause neuronal damage, and to inside joints because it does not cause tissue damage.

In an embodiment of the pain alleviation method 200, a PRF signal may be applied (e.g., using a monopolar electrode needle, such as a Stryker 20 gauge electrode needle with a 10 mm active tip, etc.) for 10 minutes at 2 Hz with a pulse width of 10 ms and 55 V at 42 degrees Celsius to an intrabursal area 10 of a joint. Other similar settings and durations maybe used.

In another embodiment of the pain alleviation method 200, the PRF signal maybe applied to multiple areas, as illustrated in FIG. 3. For example, a first PRF signal may be applied to the intrabursal area for 10 minutes at 2 Hz with a pulse width of 10 ms and 55 V at 42 degrees Celsius, while a second concomitant PRF signal maybe applied to an intraarticular area 11 of the joint for 10 minutes at 2 Hz with a pulse width of 10 ms and 55 V at 42 degrees Celsius. Other similar settings and durations maybe used.

The application of the multiple PRF signals may be simultaneous. The temperatures may be set up to 42 degrees Celsius or any temperature between 40-42 degrees Celsius. Alternatively, the application of the multiple PRF signals may be in multiple different respective intrabursal areas 10. The application of the multiple PRF signals may be in multiple different respective intrabursal 10 and intraarticular 11 areas.

Embodiments of the pain alleviation method 200 described herein may be applied using existing medical devices. For example, an embodiment of the pain-alleviation method 200 maybe applied using multiple PRF signals (see FIG. 3).

While embodiments of the pain alleviation method 200 described herein may be applied using existing medical devices, other embodiments of a new pain-alleviation device 50 are described later. Embodiments of the pain-alleviation method 200 described herein may also use other methods of ascertaining the proper intra joint or intrabursal position for application of the PRF signal, such as using a video camera that is commonly used during arthroscopy. Other methods of preparing the patient maybe used. Also, one or more PRF signals maybe directed to one or more locations in an intraarticular and/or bursa area.

In the embodiment of the pain alleviation method 200, which is illustrated in FIGS. 2A-2F, FIG. 2A shows that an incision may be made to a joint area (202). A cannula 20 may be inserted to provide access to the inside of the joint (204, FIG. 2A, FIGS. 1A-1B). The cannula 20 may provide access to the inside of the joint for medical devices to pass through an opening space 24 of the cannula 20.

Another cannula (not shown) may be inserted at a second incision to allow for access by a second medical device, such as an endoscope (e.g., arthroscope) (not shown). Alternatively, the single cannula 20 may be used to allow access for one or more medical devices. For example, cannula 20 may be used to allow access via an arthroscope, and a surgical instrument, and a pain-alleviating medical device 50 (e.g., 50a, 50b, 50c) as described herein. A surgical instrument includes any tool that performs such functions as cutting, dissecting, grasping, holding, retracting, or suturing. A cannula 20 may provide access to one or more medical devices simultaneously or intermittently depending upon the size of the devices and the cannula. Medical devices include embodiments of a pain alleviation device 50 (e.g., 50a, 50b, 50c) to be described in more detail herein with reference to FIGS. 2A-2F, 3, 4, 5A-5D.

FIGS. 2A-2F also illustrate an embodiment of a pain-alleviation medical device 50a that may be used to apply an embodiment of the pain-alleviation method 200 described herein. FIG. 2B illustrates inserting a pain-alleviation medical device 50a through the cannula 20 (206), which may be performed by using a catheter 22. The catheter 22 may be placed so that a PRF signal of the pain-alleviation device 50 may reach an intraarticular and/or intrabursal area 10 of a joint 1. The pain-alleviation medical device 50a may apply a PRF signal to one or more areas in the intrabursal area (206) at or around the settings described herein, e.g., 10 minutes at 2 Hz with a pulse width of 10 ms and 55 V at 42 degrees Celsius.

FIG. 2C illustrates the cannula 20 being removed from the joint 1, leaving the pain-alleviation medical device 50a within the intraarticular and/or intrabursal area 10 of the joint 1 (208). In this embodiment, the pain-alleviation medical device 50a includes a flexible catheter 22. FIG. 2D illustrates how the incision may be sutured to help keep the catheter 22 that supports the pain-alleviation medical device 50a in place (210), further steri-strip or another adhesive device may help immobilize the catheter. As described above, the pain-alleviation method 200, including applying a PRF signal according to the settings described herein, may have been initiated before, during, or after surgery. The PRF signal may not be initiated until after surgery, whereby the pain-alleviation device 50a may be attached to a PRF generator 40 and the signal may be applied to the intraarticular and/or intrabursal area 10 (212) at or around the settings described herein.

As illustrated in FIG. 2E, to better control how the PRF signal is delivered to the intraarticular and/or intrabursal area 10, the pain alleviation device 50a may include a suction device that may perform suctioning (212) to improve the PRF signal delivery by removing any fluid, blood, or other materials in the intraarticular and/or the intrabursal area 10, at a relative constant level to provide or direct the PRF signal to a nerve or nerve-endings, or to the inner portions of the joint or bursal tissue (214).

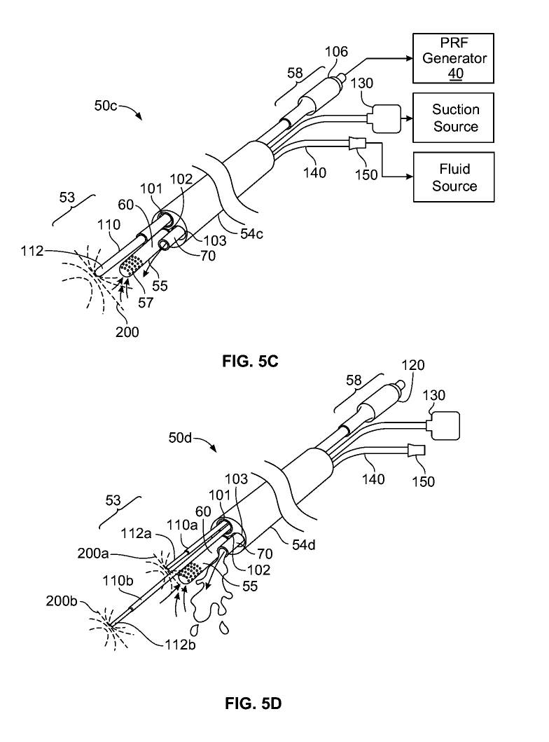

To assist in the controlling the PRF signal delivery, the method 200 will include removing fluid from the joint or associated area 10 (later described in more detail with reference to FIG. 5B, 5D). After the treatment cycle is performed (e.g., in accordance with settings described herein), the pain-alleviation (PA) and cartilage sparing device 50a may be removed (216) (FIG. 2F).

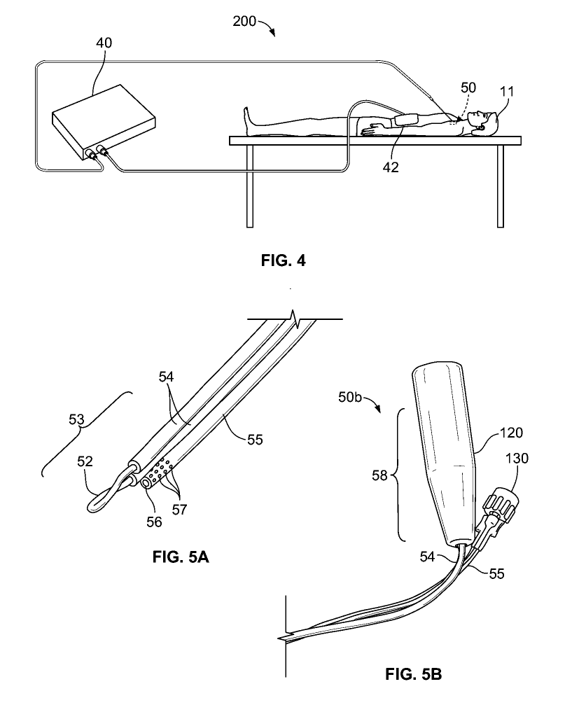

To better understand how an embodiment of a pain-alleviation and cartilage sparing device 50 may perform its objectives, FIG. 4 illustrates an overview of how an embodiment of a pain-alleviation device 50 may be integrated with a PRF generating source 40 and a return electrode 42 to apply embodiments of the pain-alleviation method 200 described herein. In particular, a PRF generating source 40 may be coupled to an embodiment of a pain-alleviation device 50 to supply a PRF signal to an intrabursal area 10 of a joint 1 of a patient 11. An example of a PRF generating source 40 is a Baylis RF generator, or a Stryker PRF generator, or Codman PRF generator, or Amp PRF generator, or NeuroTherm generator, or a similar product that maybe used to provide the PRF settings described herein.

Referring to FIGS. 4 and 5A-5B, 2E, a PRF electricity-generating source 40 may supply electricity to an embodiment of a pain-alleviation device 50 (e.g., 50a, 50b) that may include an insulated active electrode 52. The distal end 53 of the electrode 52 may not be insulated, as it is situated in or near the vicinity of a target tissue, such as nerve tissue in the intrabursal area 10 of a joint 1. When the PRF generating source 40 is turned on, current may flow from the PRF generating source 40 to the target, as the electrical impedance of the surrounding tissue may permit the flow of current. It has been suggested that the geometry and tissues of the joint (not limited to but including shoulder, elbow, wrist, ankle, knee, and hip) can prevent the loss of electrical current by reflecting the pulsed radiofrequency and keeping it concentrated in the joint. The voltage of the generator 40 may be established between an active electrode 52 and a dispersive electrode, a grounding pad 42 (FIG. 4) that may be placed on the patient's arm or leg. Body tissues complete the circuit, and the RF current flows through the tissue, producing an electric field that produces the modulation effect on pain, cartilage and blood vessels as well as other tissues.

Regardless of the exact mechanisms that cause the pain to be alleviated, embodiments of the described pain alleviation device 50 may be used towards alleviating pain. FIG. 5A illustrates another embodiment of a pain-alleviation device, 50b. A pain-alleviation device 50b may include a flexible insulating tube 54 that may support a suction tubing 55 and an active electrode 52. In this embodiment the tip opening 56 of the flexible insulation tube 54 may have fenestrations (multiple openings) 57 which are configured to allow for better control of suction (and prevent clogging of a single opening) by the suction device 60 using suction tubing 55 that may be inside the flexible insulation tube 54. Other configurations, wherein the suction tubing 55 runs parallel to the flexible insulation tube 54, instead of the suction tubing 55 being contained part of the way or totally inside the flexible tubing 54 may also be used.

For example, a connection end 58 of an embodiment of the pain-alleviation device 50b is illustrated in FIG. 5B. The flexible insulation tube 54 is shown as being separate from the suction tubing 55 of the suction device 60. Suction tubing 55 includes a suction device connection 130 that may be coupled to a suction canister or other appropriate suction device. Flexible insulation tube 54 includes a PRF connector 120 that may be coupled to a PRF generator that provide PRF signal delivery in accordance with the setting described herein.

FIG. 5C illustrates another embodiment of pain alleviation device, 50c, wherein tube 54 of embodiment 50bm may be substituted for a multiple-lumen flexible tube structure 54c, e.g., short catheter, flexible catheter, or a similar structure. In any event, the pain alleviation device 50 may use different structures, as long as the structure includes a separate lumen to support different and separate functionality. Current PRF devices can deliver PRF energy to up to 4 separate sites.

For example, as illustrated in FIG. 5C, a first lumen 101 may support an insulated PRF electrode 110 that has an exposed active tip 112. The first lumen 101 may allow for passage of straight or curved PRF introducer cannula (not shown) that may have a sharp or blunt tip, and which may help extend or enable better placement of a PRF electrode 110. In another embodiment, a PRF introducer cannula may be attached toward the distal end of the flexible tube catheter 54c.

Other embodiments may also be used. For example, the lumen 101 may support a flexible PRF electrode 110, which may not require an introducer cannula. The PRF electrode 110 maybe reusable or disposable. Like shown in FIG. 5B, the connection end 58 of the PRF electrode 110 may have a connector 120 that connects it to a PRF generator 40, or the PRF electrode 110 may have a longer length and a direct connection 106 that interfaces with a port of a PRG generator 40. The PRF electrode 110 may come in various sizes and shapes that allow for flexible surgical positioning. The PRF electrode 110 may have a tip that includes a thermocouple sensor to monitor PRF signal out put and temperature in the area near the electrode tip. Other PRF electrode embodiments 110 may also be used. For example, a steerable electrode may also be used.

FIG. 5C illustrates embodiment 50c as having a second lumen 102 that may support suction tubing 55 that couples to a suction device (not shown) via a suction connector 130. Typically, during surgery, suction may be used to remove blood, tissues, liquids, etc. from the area being operated on to allow surgeons to view and work on the area. In an embodiment of the pain-alleviation device 50 (50a, 50b, etc.) described herein, may provide suction for an additional or different reason, which is at to, at any time: before, during, or after surgery, to use suction device 60 to assist in controlling the output signal of the PRF electrode 110 by maintaining a controlled amount of fluid in the operational area, such as the intraarticular and or intrabursal area 10. Fluid from the joint may be removed, that was originally placed or accumulated during arthroscopy or other joint surgeries.

In another embodiment, as illustrated in FIG. 5D, multiple PRF electrodes 110a, 110b maybe passed through a single lumen 101 or additional separate lumen (not shown). These embodiments may allow for additional control of a total signal 200, which may be a combination of multiple individual signals, e.g., 200a, 200b. The combination of signals may be summed or kept separate in efforts towards alleviating pain in the intraarticular and/or intrabursal region 10 or other regions.

The pain-alleviation device 50d may also include more lumens for other functional devices, such as a camera and light source device, etc. Alternatively, some of the components described herein as being co-located in the flexible tube structure 54b maybe located separately.

For example, with reference to FIG. 2A, separate surgical cannulas 20, 20 may have been used to place a first PRF electrode 110a and a second PRF electrode 110b to allow for their later placement, as is shown in FIG. 3. For example, as described above, a first PRF signal 200c may be applied to the intrabursal area for 10 minutes at 2 Hz with a pulse width of 10 ms and 55 V at 42 degrees Celsius, while a second PRF signal 200d may be applied to an intraarticular area of the joint for 10 minutes at 2 Hz with a pulse width of 10 ms and 55 V at 42 degrees Celsius.

Embodiments of the herein described pain-alleviation method and pain-alleviation device may produce results, such as the following example results.

Results from case series reported by the inventors (submitted for publication) that use embodiments described herein, support previous case reports that have shown PRF treatment improves pain and range of motion (ROM) sustained beyond conventional shoulder pain management treatment modalities. The case study does not report the use of PRF in the immediate post-operative period, nor does it report the combination of a suction device with PRF.

As a minimally invasive method with a remarkable margin of safety, embodiments of PRF methods and devices described herein are a favorable treatment alternatives for pain management, both pre- and post-operatively, for patients who: (a) do not respond to conservative treatment, (b) are poor candidates for surgical interventions to relieve pain (e.g., elderly with multiple comorbidities, patients who are unfit for anesthesia), (c) prefer non-surgical treatment options for pain management, (d) have contraindications for oral pharmacological treatment (e.g., gastric ulcers, allergies, history of substance abuse and dependency), and/or (e) have poor compliance adhering to prescribed pain management therapies that require repeated and more active engagement over time (e.g., extended physical therapy, polypharmacy regimens).

Although a definitive mechanism of action for PRF remains unclear, there are extensive ongoing research projects and publications in this area. From the emerging evidence, PRF appears to have genuine biological effects in cell morphology, synaptic transmission, pain signaling, increased blood flow and chondro sparring (cartilage preserving) effects. Moreover, PRF energy has been shown to accelerate impaired wound healing primarily through wound contraction by means of stimulating cell proliferation, granulation tissue formation, increased vascularity and collagen deposition. Another mechanism of action for PRF is a possible cartilage-protective or regenerative process. Multiple in vitro and animal studies have demonstrated that chondrocyte proliferation and matrix synthesis can be significantly enhanced by pulsed radio frequency electric field exposure.

It appears that PRF combines an anabolic effect on chondrocytes, a stimulatory effect on anabolic cytokine production, and a counteraction of inflammatory processes by anti inflammatory effects. In addition, PRF has been shown to stimulate the process of angiogenesis, leading to neovascularization in vivo studies, an important factor in healing.

Optimal procedural positioning of PRF currents is also under investigation for chronic joint pain. Some researchers have argued that intraarticular placement is best for the chronic joint pain. This concept is based on the insulating properties of bone. When the active part of an electrode is situated in soft tissue, the current intensity, and therefore, the electric field, is rapidly diluted at increasing distances from the electrode. However within a joint, part of the current is deflected by the bony surfaces, forcing it to remain inside the joint space such that dilution cannot occur to the same extent. Thus, current intensities and electric fields are higher than would be expected at a greater distance from the electrode. Alternatively, results from our case series suggests that intrabursal placement of the PRF electrode needle within the subacromial space has utility for managing chronic shoulder pain. Our empirical observations could possibly reflect an action of electric fields on immune cells, thus influencing the production of anti-inflammatory cytokines which have been reported in the subacromial bursa as well as other effects.

Our case report has possible limitation due to the small number of case (3 patients). Further, possible placebo effect maybe present. However despite these limitations, to the best of our knowledge, our study is the first to provide longer-term results specific to pain and mobility outcomes following PRF treatment for orthopaedic-related diagnoses as well as to present an innovative technique for PRF application to the betterment of patient care. Previous case-studies including successful chronic joint pain treatment with similar results to our report have been published.

I claim:

1. A method for pain alleviation, cartilage sparring and/or cartilage repair in the intraoperative and/or perioperative treatment period comprising:
verifying the location of a PRF electrode having a flexible catheter in an intraarticular and/or intracapsular and/or intrabursal area of a joint by the use of intraoperative arthroscopy;
applying a PRF signal via the PRF electrode in the intraarticular and/or intrabursal area of the joint; and
controlling fluids in the intraarticular and/or intrabursal area of the joint by removing the fluids via suction catheter in the intraarticular intracapsular and/or intrabursal area of the joint.

2. The pain alleviation method of claim 1, wherein a PRF signal is applied to the electrode for approximately 10 minutes at 2 Hz with a pulse width of approximately 10 ms and approximately 55 V at approximately 42 degrees or similar settings.

3. A device useful for pain alleviation, cartilage sparring and/or cartilage repair in the intraoperative and/or perioperative time of treatment comprising:
(a) a PRF electrode capable of being applied to an intra-articular and/or intracapsular and/or intrabursal area of a joint; and
(b) a suction device, wherein the suction device may be used to control the amount of fluid in the intraarticular and/or intracapsular and/or intrabursal area of the joint.

4. The device of claim 3, further comprising a fluid placement device, wherein the fluid placement device may be used to control the amount of fluid in the intrabursal intracapsular and/or intraarticular area of a joint in combination with the suction device either manually or automatically to direct a signal of the PRF electrode to a nerve in the intrabursal and/or intracapsular and/or intraarticulararea.

5. The device of claim 3 wherein the PRF electrode and suction device are co-located in a flexible tube structure.

6. The device of claim 5 further comprising a cannula for introducing the device into a intraarticular and/or intrabursal area of the joint.

7. The device of claim 3 wherein the suction device comprises a tube with fenestrations along its length.

8. The device of claim 3 wherein the PRF electrode is capable of applying a signal at 2 Hz with a pulse width of approximately 10 ms and approximately 55 V at approximately 42 degrees or similar settings.
